(19) United States
(12) Patent Application Publication
(10) Pub. No.: US 2003/0113819 A1
(43) Pub. Date: Jun. 19, 2003

(54) Carrier peptide and method for delivery of molecules into cells

(76) Inventors: Jeffrey K Horton, Vale of Glamorgan (GB); John a Smith, Cardiff (GB); Michelle L Teear, Cardiff (GB); Jonathan M Kendall, Gwent (GB); Nigel P Michael, Swansea (GB)

Correspondence Address: AMERSHAM BIOSCIENCES, PATENT DEPARTMENT, 800 CENTENNIAL AVENUE, PISCATAWAY, NJ, 08855, US

(21) Appl. No.: 10/148,936

(22) PCT Filed: Dec. 4, 2000
(86) PCT No.: PCT/GB00/04593

(51) Int. Cl. G01N 33/574; C07K 7/08; C07K 7/06

(52) U.S. Cl. 435/7.23; 530/327; 530/328

(57) ABSTRACT

Disclosed is a carrier peptide and a conjugate comprising a carrier peptide and a target molecule and a method for delivery of the target molecule into a cell. The carrier peptide contains from 10-15 amino acids, having a core sequence of 3-5 hydrophobic amino acids flanked by flanking amino acid sequences. The core sequence comprises residues selected from proline and leucine such that there is at least one each of proline and leucine and is symmetrical about an amino acid or a bond. Also disclosed is a method for delivery of a target molecule into a cell and a method for measuring a cellular process.

The present invention relates to the delivery of molecules, for example, proteins, peptides, enzymes, carbohydrates, nucleic acids, reporter groups, drugs and hormones, into cells. In particular, the invention relates to new peptide sequences and methods employing such peptides for delivering molecules into cells.

Many biological molecules and their analogues, including peptides, proteins, nucleic acids, or exogenous substances, such as drugs and hormones are preferably incorporated within the cell in order to produce their effect. Internalisation of biomolecules by living cells offers a powerful tool for studying cellular function. However, for many such molecules, the cell membrane presents a selective barrier, which is generally impermeable to polar and charged molecules. Consequently, the incorporation of specific proteins, nucleic acids or other biomolecules into cells must be facilitated by various delivery methods. There are various conventional methods for cell delivery, including permeabilisation of the cell membrane, microinjection into the cell, and electroporation. Others include the use of viral vectors, chemical methods, bacterial toxins and liposome techniques.

A more recent approach to deliver specific peptide sequences into cells has been through the use of signal peptide sequences as a carrier vehicle. Signal peptides share a common core motif, which is hydrophobic in character, and they are capable of mediating translocation of secretory proteins across the cell membrane. U.S. Pat. No. 5,807,746 discloses a method for importing biologically active molecules, such as peptides, nucleic acids, carbohydrates, lipids and therapeutic agents, into a cell by administering a complex comprising the molecule to be imported, linked to an importation competent signal peptide. Rojas et al (Nature Biotechnology, 16, 370-375, 1998) describes the attachment of a membrane translocating sequence (MTS) to proteins up to 41 kDa. MTS is a specific peptide sequence of twelve amino acids from the hydrophobic region of the signal sequence of Kaposi fibroblast growth factor.

Hawiger et al (Curr. Opinion Chem. Biol., (1999), 89-94) describes methods for the delivery of functional peptides and proteins into cells, based on the cell membrane permeable properties of the hydrophobic region of a signal peptide sequence.

WO 99/05302 discloses novel constructs of peptides and nucleic acid analogues, which are conjugated together for delivery to intracellular components such as RNA, DNA, enzymes, receptors and regulatory elements.

WO 99/64455 discloses DNAs encoding peptides having nuclear transport activity, by taking advantage of the properties of a transcription factor.

WO 97/12912 discloses a peptide sequence containing sixteen amino acids comprising between six and ten hydrophobic amino acids and containing tryptophan at position six.

WO 99/05302 discloses constructs of specific peptide sequences and nucleic acid analogues conjugated together for transport across a lipid membrane of a cell and for delivery into contact with intracellular components, such as nucleic acids, enzymes and receptors.

Canadian patent application No. 2094658 describes the intracellular delivery of biochemical agents, such as therapeutic peptides and oligonucleotides, facilitated by a coupled carrier peptide consisting of positively charged amino acids. In a preferred embodiment, the peptides consists of eight or nine D-arginine residues.

The prior art methods described above are useful in specific experimental situations but may have drawbacks as generic methods. For example, microinjection may be applied only where a single cell or a very few cells are being studied. Bacterial toxins cannot deliver high concentrations of biomolecules into cells without killing target cells when a lethal amount of activity is employed. In contrast, the use of carrier peptides to deliver biomolecules and other chemical compounds into cells potentially offers a more generic approach which may be adapted for a range of applications. Accordingly, there is a need to develop new reagents that are useful for the transport of biologically active molecules and other chemicals into cells without disrupting the cellular metabolism or damaging the target cells. This need is addressed by the present invention which relates to a peptide reagent, which, when coupled to a target molecule facilitates transport of the target molecule across the cell membrane and into the cell.

According to one aspect of the present invention, there is provided a carrier peptide for transport of a target molecule across a cell membrane and into a cell, the carrier peptide having from 10-15 amino acids and having a core sequence of 3-5 hydrophobic amino acids flanked by flanking amino acid sequences, characterised in that said core sequence comprises residues selected from proline and leucine such that there is at least one of each of proline and leucine and that the core sequence is symmetrical about an amino acid or a bond.

According to a second aspect of the invention, there is provided a conjugate comprising a carrier peptide linked by means of a covalent bond to a target molecule for delivery of the target molecule into a cell, wherein the carrier peptide contains from 10-15 amino acids and comprises a core sequence of 3-5 hydrophobic amino acids flanked by flanking amino acid sequences, characterised in that said core sequence comprises residues selected from proline and leucine such that there is at least one of each of proline and leucine and that the core sequence is symmetrical about an amino acid or a bond.

In the first and second aspects of the invention, suitably, the core sequence of amino acids is selected from proline and leucine and is symmetrical about an amino acid or a bond. By the term “symmetrical about an amino acid or a bond” it is meant that the amino acid residues covalently linked on either side of at least one core amino acid, or alternatively, the bond joining two core amino acid residues, are the same. Suitably, the core sequences of amino acids according to the invention are selected from: -Pro-Leu-Pro-; Leu-Pro-Leu-; -Pro-Leu-Leu-Pro-; -Leu-Pro-Pro-Leu-; -Pro-Pro-Leu-Pro-Pro-; -Leu-Leu-Pro-Leu-Leu-; -Pro-Leu-Pro-Leu-Pro- and -Leu-Pro-Leu-Pro-Leu-. Particularly preferred core sequences are selected from:

-Pro-Leu-Pro-;

-Leu-Pro-Pro-Leu-

-Pro-Leu-Leu-Pro-;

-Pro-Pro-Leu-Pro-Pro-.

It is to be understood that the amino acids making up the core sequence may also include D-isomers of the amino acids.

The sequences flanking the core sequence of amino acids are typically each independently from 3-7 amino acids in length and may be selected from naturally occurring L-amino acids, for example: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or Y), tryptophan (Trp or W), tyrosine (Tyr or Y) and valine (Val or V). It is to be understood that the 3-7 amino acids flanking the core sequence of amino acids are not limited to the examples described above and may be represented by analogues of amino acids, including D-amino acids.

In a preferred embodiment of the first aspect, carrier peptides for use in the present invention are selected from the group consisting of:

(SEQ ID NO:1)

Thr-Lys-Lys-Pro-Leu-Pro-Thr-Pro-Glu-Glu-Asp-

(SEQ ID NO:2)

Ser-Glu-Pro-Ala-Val-Ser-Pro-Leu-Leu-Pro-Arg-Lys-
Glu-Arg

(SEQ ID NO:3)

Ala-Pro-Thr-Met-Pro-Pro-Pro-Leu-Pro-Pro-Leu-Gly-
Gly-Lys

(SEQ ID NO:4)

Ala-Pro-Thr-Arg-Val-Pro-Leu-Pro-Leu-Pro-Val-Gly-
Gly-Lys

(SEQ ID NO:5)

Ala-Pro-Thr-Arg-Ala-Val-Leu-Pro-Leu-Ala-Val-Gly-
Gly-Lys

(SEQ ID NO:6)

Ala-Pro-Thr-Arg-Val-leu-Pro-Pro-Leu-Val-Ala-Gly-
Gly-Lys;

(SEQ ID NO:7)

Ala-Pro0Thr-Arg-Val-Leu-Leu-Pro-Leu-Leu-Val-Gly-
Gly-Lys

(SEQ ID NO:8)

and Ala-Pro-Thr-Arg-Leu-Pro-Leu-Pro-Leu-Val-Ala-
Gly-Gly-Lys

In a second preferred embodiment of the first aspect, the DNA sequences encoding the carrier peptides according to Sequence ID Nos. 1 to 8 are as follows:

(SEQ ID NO:9)

5′-ACT-AGG-AAG-CCT-CTT-CCT-CCT-ACT-CCT-GAG-GAG-
GAT-3′;

(SEQ ID NO:10)

5′-TCT-GAG-CCT-GCT-GTT-TCT-CCT-CTT-CTT-CCT-CGT-
AAG-GAG-CGT-3′;

(SEQ ID NO:11)

5′-GCT-CCT-ACT-ATG-CCT-CCT-CCT-CTT-CCT-CCT-CTT-
GGT-GGT-AGG-3′;

(SEQ ID NO:12)

5′-GCT-CCT-ACT-CGT-GTT-CCT-CTT-CCT-CTT-CCT-GTT-
GGT-GGT-AAG-3′;

(SEQ ID NO:13)

5′-GCT-CCT-ACT-CGT-GCT-GTT-CTT-CCT-CTT-GCT-GTT-
GGT-GGT-AAG-3′;

(SEQ ID NO:14)

5′-GCT-CCT-ACT-CGT-GTT-CTT-CCT-CCT-CTT-GTT-GCT-
GGT-GGT-AAG-3′;

(SEQ ID NO:15)

5′-GCT-CCT-ACT-CGT-GTT-CTT-CTT-CCT-CTT-CTT-GTT-
GGT-GGT-AAG-3′; and

(SEQ ID NO:16)

5′-GCT-CCT-ACT-CGT-CTT-CCT-CTT-CCT-GTT-GTT-GCT-
GGT-GGT-AAG-3′.

As is well known in the art, a single amino acid may be encoded by more than one nucleotide codon and thus each of the above nucleotide sequences may be modified to produce an alternative nucleotide sequence that encodes the same peptide. Thus, the preferred embodiments of the invention include alternative DNA sequences that encode the preferred peptide sequences as previously described. It is to be understood that the preferred amino acid and nucleic acid sequences may include additional residues, particularly N- and C-terminal amino acids, or 5′- or 3′-nucleotide sequences, and still be essentially as described herein, as long as the peptide sequence facilitates transport of the target molecule across the cell membrane and into the cell.

Suitably, the target molecule may be a reporter moiety, preferably a radioactively-labelled moiety or a luminescent molecule. Alternatively, the target molecule may be a biological molecule. Optionally, the conjugate comprising a carrier peptide linked to a biological molecule may additionally contain a reporter moiety covalently bonded thereto, the reporter moiety being as defined hereinbefore.

Suitably, the radioactively-labelled moiety will be labelled with a radioisotope that emits β-particles or electrons having a mean free path of up to 2000 μm in aqueous media. Suitable radioisotopes are those commonly used for labelling biomolecules and used in biochemical applications and include ¹⁴C, ³H, 35S, ³³P, ¹²⁵I, ³²P, ⁴⁵Ca, ⁵⁵Fe, ⁵¹Cr, ⁸⁶Rb and ¹⁰⁹Cd.

Suitable luminescent molecules include fluorescent dyes selected from the group consisting of fluoresceins, rhodamines, coumarins, pyrene dyes and cyanine dyes. For example, the luminescent molecule may be an environmentally sensitive fluorescent dye, such as a Ca²⁺-sensitive indicator, (eg. Fluo-3, Fura-2 and Quin-2), a pH sensitive probe, (eg. carboxy-SNARF®-1 dye (Molecular Probes Inc)), or the fluorescent dye may be capable of being modified by enzyme activity, leading to a change in fluorescent properties of the dye. For example, there are phosphate probes which can detect the activity of kinases and phosphatases e.g. dimethylacridinone (DDAO, available from Molecular Probes Inc.). Such fluorescent probes may be linked to the carrier peptide by means of a linking group which is cleavable by intracellular esterases upon transport of the probe across the cell membrane and into the cell. Alternatively, the luminescent molecule may be a fluorescent or a bioluminescent protein, such as Green fluorescent protein (GFP) and analogues thereof, a photoprotein such as aequorin, or a luciferase.

Suitable biological molecules may be selected from the group consisting of antibodies, antigens, proteins, enzymes, carbohydrates, lipids, drugs, hormones and nucleotides which contain or are derivatised to contain one of amino, hydroxyl, phosphate, thiophosphoryl, sulphydryl, aldehyde or carboxyl groups and deoxy- or ribo-nucleic acids (such as DNA or RNA) which contain or are derivatised to contain one of amino, hydroxyl, phosphate, thiophosphoryl, sulphydryl, aldehyde or carboxyl groups.

In one embodiment, the carrier peptide may be chemically conjugated to the target molecule by means of covalent attachment between a target bonding group on the carrier peptide and a complementary functional group on the target molecule. The target bonding group can be any group suitable for covalently attaching the carrier peptide to the target molecule and methods for forming a covalent linkage will be well known to those skilled in the art. In a preferred aspect of the present invention, the covalent bond linking the carrier peptide and the target molecule is labile to the extent that the target molecule may be cleavable and thereby separated from the carrier peptide once the target molecule has been transported into the cellular environment. Examples of cleavable linkage groups include a disulphide linkage, which may be cleaved upon reaction with a cytosolic enzyme such as glutathione reductase in the presence of NADPH, and an ester linkage, which may be cleaved by non-specific cellular esterases present in the cytosol.

Suitably, the target bonding group may be a terminal amino group or a terminal carboxyl group of the carrier peptide. Alternatively, the target bonding group may be a functional group located on a non-terminal amino acid. Suitable non-terminal target bonding groups include the ε-amino group (present in lysine), carboxylic acid groups (present in aspartic acid and glutamic acid) and the thiol group (present in cysteine). Where it is required to couple the carrier peptide to the target molecule through the terminal or non-terminal target bonding group of the carrier peptide, it is desirable that the target molecule should contain a complementary functional group capable of reacting with the amino, carboxyl, or thiol groups under suitable reaction conditions.

For example, the carrier peptide may be coupled to the target molecule through the formation of a disulphide linkage between the thiol group of a cysteine residue in the carrier peptide and a thiol group in the target molecule. Thiol-thiol coupling may be achieved by atmospheric oxidation or by employing other oxidising agents such as potassium ferricyanide. Target molecules which already contain a disulphide link may be treated with a reducing agent such as dithiothreitol or β-mercaptoethanol in order to generate a thiol group, prior to coupling with the carrier peptide. Alternatively, the terminal carboxyl group of the carrier peptide may be coupled to the ε-amino group of a lysine residue contained in a peptide or protein target molecule, by means of a water soluble coupling agent such as 1-ethyl-3-[3-dimethyl aminopropyl] carbodiimide hydrochloride (EDC). Care must be taken in these approaches to avoid self-coupling of either the carrier peptide or the target molecule.

Carbohydrate-containing target molecules may be treated with an oxidising agent such as periodic acid and the resultant aldehyde residues reacted with amino groups on the carrier peptide. The resultant Schiff's base may be stabilised by treatment with a suitable reducing agent such as sodium cyanoborohydride. Other methods of linking the carrier peptide to the target molecule may utilise enzymatic coupling using enzymes such as transglutaminase and carboxypeptidase.

Alternatively, it may be desirable to conjugate the carrier peptide to the target molecule indirectly, through the use of a chemical cross-linking reagent. Numerous cross-linking methods are known and potentially applicable for conjugating the carrier peptides described herein to target molecules. However, many known chemical cross-linking methods are not specific, that is, they do not direct the point of coupling to any particular site on the carrier peptide or target molecule. As a result, use of non-specific cross-linking agents may attack or sterically block active sites, thereby rendering the conjugated proteins biologically inactive. A preferred method for increasing the specificity of coupling is to direct the chemical coupling to a functional group found only once or a few times in the peptide to be cross-linked, for example, a thiol group of cysteine. In the case where a carrier peptide contains no lysine residues, use of a cross-linking reagent specific for primary amines will be selective for the terminal amino group of the carrier peptide. Successful utilisation of this approach to increase coupling specificity requires that the biomolecule has suitable functional groups at positions that may be chemically altered without loss of biological activity.

Cross-linking reagents may be homo-bifunctional, that is having two functional groups that undergo the same reaction. An example of a homo-bifunctional cross-linking reagent is bis-maleimido hexane (BMH), containing two maleimido-groups, which react specifically with, and link, thiol-containing compounds under mild conditions (pH 6.5-7.7). BMH is useful for linking peptides or proteins that contain cysteine residues. Cross-linking reagents may also be hetero-bifunctional, that is having two different functional groups, for example an amine-reactive group and a thiol-reactive group. Suitable hetero-bifunctional cross-linking agents include succinimidyl 4-(N-maleimidomethyl) cyclohexane-1-carboxylate (SMCC), m-maleimido-benzoyl-N-hydroxysuccinimide ester (MBS), and succinimidyl 4-(p-maleimidophenyl) butyrate (MPB). The succinimidyl group of the above cross-linking reagents reacts with a primary amine, and the maleimido group forms a covalent bond with the free thiol group of a cysteine residue. Another example is N-5-azido-2-nitrobenzoyloxysuccinimide (ANB-NOS). The succinimide group reacts with primary amino groups, whilst subsequent photolysis at 320-350 nm causes non-specific labelling of a second molecule.

In cases where the target molecule does not contain a free thiol group for reaction with the target bonding group of the carrier peptide, it may be desirable to introduce a thiol group, either by cleavage of a protein dithio (—S—S—) linkage, or by protein thiolation prior to the coupling reaction. Suitable thiolation reagents include N-succinimidyl S-acetylthioacetate (SATA), N-acetyl-DL-homocysteine thiolactone (AHTL) and S-acetyl-mercaptosuccinic anhydride (SAMSA). Furthermore, methods are available for thiolation via carboxyl groups, aldehyde groups and hydroxyl groups. To increase aqueous solubility, the cross-linking reagent may include a water solubilising group, such as sulphonate. Suitable water soluble reagents include sulpho-MBS and sulpho-SMCC. In some cases, it may be advantageous to employ a cleavable cross-linking reagent that can be cleaved by non-specific cellular esterases that are common in the cell cytosol. The use of a cleavable cross-linking reagent permits the target molecule to be cleaved from the carrier peptide after delivery into the target cell. Direct disulphide linkages may be useful in the invention described herein; alternatively cross-linkers such as N-γ-maleimidobutyryloxy-succinimide ester (GMBS) and sulpho-GMBS have reduced immunogenicity. In some aspects of the present invention, such reduced immunogenicity will be advantageous. Techniques for cross-linking the carrier peptide with the target molecule will be well known to the skilled person, (see for example Wong, S. S., Chemistry of Protein Conjugation and Cross-Linking, CRC Press (1991); Aslam, M. and Dent, A. (Eds) Bioconjugation: Protein Coupling Techniques for the Biomedical Sciences. Macmillan Press (1998).

The chemical coupling of the carrier peptides of the present invention to the target molecule may be accomplished with a target molecule having at least one functional group as described hereinbefore, suitable for reaction under appropriate conditions with a target bonding group of the carrier peptide. The target molecule, or a derivative thereof, and a carrier peptide according to the present invention are incubated under conditions and in appropriate amounts, for a period of time sufficient to permit the target molecule to react with and covalently bond to the carrier peptide. The extent of coupling, that is the number of carrier peptide units per target molecule must be controlled by careful adjustment of reaction conditions such as pH, molar ratio of reactants and the concentration of reactants.

In another embodiment, the carrier peptide may be produced in a fusion protein with a peptide or protein target molecule. Using the sequence information described, the carrier peptide can be produced by recombinant DNA methodology. See for example, Sambrook, J. et al (1989) Molecular Cloning—A Laboratory Manual, Cold Spring Harbor Laboratory Press. Furthermore, the carrier peptide sequence can be joined in-frame with a target molecule sequence of interest and the desired fusion protein produced when inserted into an appropriate expression vector. For example, polymerase chain reaction or complementary oligonucleotides can be employed to engineer a polynucleotide sequence corresponding to the carrier peptide sequence, 5′ or 3′ to the gene sequence corresponding to the target peptide or protein of interest. Alternatively, the same techniques can be used to engineer a polynucleotide sequence corresponding to the carrier peptide sequence 5′ or 3′ to the multiple cloning site of an expression vector prior to insertion of a gene sequence encoding the target protein of interest. The polynucleotide sequence corresponding to the carrier peptide sequence may comprise additional nucleotide sequences to include cloning sites, linkers, transcription and translation initiation and/or termination signals, labelling and purification tags.

Expression of the engineered polynucleotide may be performed utilising a wide variety of expression systems that are commercially available for recombinant protein production. Suitable cloning vectors and host cells may be selected from prokaryotic (Unger, T. F., The Scientist 11 (17), 20-23, 1997); yeast, insect and plant (Smith, C., The Scientist 12 (22): 20, 1998); and mammalian (Smith, C., The Scientist 12 (3): 18, 1998). A number of issues have to be considered when selecting a suitable expression system. See, for example, the table comparing desired characteristics with each expression system provided in Fernandez, J. M. & Hoeffler, J. P., Gene Expression Systems—using nature for the art of expression, Academic Press (1999), page 4. For example, a eukaryotic system would prove a suitable choice for proteins requiring post-translational modification.

The expression systems mainly comprise plasmid or virion-plasmid hybrid vectors which may contain transcriptional and translational regulatory elements, protein targeting signals, multiple cloning sites, fusion tags, selection markers and replication elements. Expression of the engineered polynucleotide is carried out when the vector, with the desired polynucleotide sequences inserted into a multiple cloning site, is introduced into a suitable host cell. Examples of different hosts include, but are not limited to, Escherichia coli for prokaryotic expression; Saccharomyces cerevisiae for yeast expression; Drosophila melanogaster for insect expression; Nicotiana tabacum for plant expression and Chinese hamster ovary cells for mammalian expression. Bacteria and yeast offer the ease of microbial growth and gene manipulation relative to the more complex eukaryotic expression systems. Following transformation, the transformed host cells are cultured in an appropriate medium suitable for cell growth and the recombinant proteins are expressed in a constitutive or inducible manner.

For example, a fusion protein comprising glutathione S-transferase (GST), carrier peptide and apoaequorin can be constructed and expressed in E. coli. The carrier peptide may be joined in-frame to the C-terminus of GST and the N-terminus of apoaequorin in a pGEX plasmid vector (Amersham Pharmacia Biotech). Recombinant production of the fusion protein is carried out utilising a standard E. coli expression host, followed by purification employing glutathione affinity chromatography and removal of the GST tag by proteolytic cleavage.

In a third aspect of the present invention, there is provided a method for delivery of a target molecule into a cell. The method comprises the steps of: providing a conjugate for delivery of the target molecule into a cell, the conjugate comprising a carrier peptide covalently bonded to a target molecule, wherein the carrier peptide contains from 10-15 amino acids and comprises a core sequence of 3-5 hydrophobic amino acids flanked by flanking amino acid sequences, characterised in that the core sequence comprises residues selected from proline and leucine such that there is at least one of each of proline and leucine and that the core sequence is symmetrical about an amino acid or a bond; and contacting the cell with the conjugate under conditions so as to effect delivery of the target molecule into the cell.

Typically, cultured cells are incubated with the conjugate at a concentration of 0.1 to 100 μM in a suitable cell culture medium under conditions suitable for cell growth and for a time which may range from 0.5 to 24 hours. Cells are cultured according to standard cell culture techniques, eg. cells are cultured in a suitable vessel in a sterile environment at 37° C. in an incubator containing a humidified 95% air/5% CO₂ atmosphere. Vessels may contain stirred or stationary cultures. Various cell culture media may be used including media containing undefined biological fluids such as foetal calf serum, as well as media which is fully defined, such as 293 SFM II serum free media (Life Technologies Ltd., Paisley, UK). There are established protocols available for the culture of diverse cell types. (See for example, Freshney, R. I., Culture of Animal Cells: A Manual of Basic Technique, 2^{nd} Edition, Alan R. Liss Inc. 1987). Typically, optimal incorporation of the conjugate occurs within about 1 to 2 hours of incubation. The method of the invention may be used with any adherent or non-adherent cell type that can be cultured in standard tissue culture plastic-ware. Such cell types include all normal and transformed cells derived from any recognised source with respect to species (eg. human, rodent, simian), tissue source (eg. brain, liver, lung, heart, kidney skin, muscle) and cell type (eg. epithelial, endothelial). In addition, cells which have been transfected with recombinant genes may also be cultured and utilised in the method of the invention.

The conjugate, comprising a carrier peptide of the present invention and the target molecule, may be transported to a particular region of the cell, for example the nucleus, when the carrier peptide is linked in-frame with a particular target sequence. When the target molecule is required to be delivered to cells grown in cell or tissue culture, the conjugate is simply added to the culture medium. This is useful as a means of delivering into the nucleus, agents whose effect on cellular processes needs to be assessed. The invention described herein will therefore be of particular value in the drug discovery process. The method of the invention may be used, but not restricted to, the delivery of target molecules such as fluorescent dyes, enzyme substrates, EGFP/GFP, chemiluminescent reporters, antibodies, antibody fragments and binding domains, transcription factors and targeted sequences. Furthermore, in vitro, the method allows for the efficient transfection of cells without carrying out cell damaging procedures. Therefore, the reagent and method described herein are useful for any process that requires transfection techniques, such as for transfecting reporter genes into cells, to screen for compounds that affect the expression of the reporter gene, or transfecting into cultured cells a gene to affect protein expression in the cells.

For in vivo applications, the molecule, for example a biomolecule, drug therapeutic or imaging agent, linked to the carrier peptide can be added to blood or tissue samples, or to a pharmaceutically acceptable carrier e.g. saline and administered by one of several means known in the art. Examples include, but are not limited to, intravenous, oral or topical administration, vaginal or rectal administration, particularly when the agents are in a suppository form. The invention described herein is not limited to drug delivery methods and can used for administration of vaccines, gene therapy, radiopharmaceuticals and as a means for producing cell-permeable proteins for the treatment of cancer.

In a fourth aspect of the invention, there is provided a method for measuring a cellular process. The method comprises providing a population of cells in a fluid medium and contacting the cells with a conjugate under conditions so as to effect delivery of the conjugate into the cells and where the conjugate comprises a carrier peptide covalently bonded to a reporter moiety, or to a biological molecule containing a reporter moiety covalently bonded thereto. The carrier peptide contains from 10-15 amino acids and comprises a core sequence of 3-5 hydrophobic amino acids flanked by flanking amino acid sequences, characterised in that the core sequence comprises residues selected from proline and leucine such that there is at least one of each of proline and leucine and that the core sequence is symmetrical about an amino acid or a bond. The cellular process is measured by detecting the output of the reporter moiety.

In a particular embodiment of the fourth aspect, the cells may be contacted with the conjugate in the presence of a substance whose effect on the cellular process is to be determined. In this embodiment, the detection step provides a measurement of the effect of the test substance on the cellular process.

By cellular process it is intended to mean one of the normal processes which living cells undergo including: biosynthesis, uptake, transport, receptor binding, metabolism, fusion, biochemical response, growth and death. The method is particularly suitable for determining the effect on a cellular process of test substance and may be applied to a compound whose metabolism and toxicology towards a particular cell type is under investigation, eg. drugs, enzyme inhibitors, antagonists and the like.

Suitably, the reporter moiety will be a luminescent molecule, in which case the detection step may be accomplished either by non-imaging counting (such as a luminometer), or alternatively, by imaging techniques, preferably by means of a cooled charge coupled device (CCD) imager (such as a scanning imager or an area imager). Imaging is quantitative and fast, and instrumentation suitable for imaging applications is now available for detecting light emissions from the whole of a multiwell plate.

The following two examples illustrate methods which may be performed for the measurement of the effect of a substance on a cellular process:

i) Measurement of Intracellular Calcium using Recombinant Aequorin

Confluent layers of tissue culture cells (HeLa or CHO) are treated with recombinant apoaequorin fused to the carrier peptide (constructed and expressed as described herein), at a concentration of 0.1-100 μM for 1-24 hours at 37° C. in complete tissue culture medium. The cells are washed extensively in PBS (or other suitable buffer), then trypsinised and seeded onto glass coverslips. For intracellular Ca²⁺ measurements, the recombinant apoaequorin transported into the cells is converted to the photoprotein by adding coelenterazine (2 μM) to the cells at least 4 hours prior to the start of the experiment. The coverslips are then inverted over the reservoir of a perfusion chamber maintained at 37° C. Light emission from the cells may be measured by CCD-based imaging (Badminton et al Journal of Biological Chemistry 271, 31210-31214, (1996)).

Cells are perfused for at least 10 minutes to remove excess coelenterazine. HeLa or CHO cells, which are known to mobilise intracellular Ca²⁺via the generation of IP₃ in response to agonists, can then be challenged with a suitable agonist, eg. histamine, ATP or bradykinin. Increases in cytosolic Ca²⁺ will be accompanied by an increase in light emission. The light emission from the cells can be converted to absolute Ca²⁺ by subtracting the fractional discharge of aequorin after addition of agonists from total light emitted by the photoprotein following exposure of the cells to 5 mM aqueous CaCl₂. This value can be converted to absolute Ca²⁺ by comparisons with suitable calibrations of the recombinant aequorin.

ii) Assay for the Determination of the Effect of an Inhibitor of Caspase Activity

This assay is suitable for the determination of the effect of an inhibitor such as staurosporine on cellular apoptosis (and thus caspase activity). The assay depends on the delivery into cells grown in culture of a fluorogenic substrate containing a cleavable (-Asp-Glu-Val-Asp-) peptide sequence (Xu, X. et al, Nucleic Acids Research, (1998), 26(8), 2034-2035). The substrate combines a fluorescent donor dye molecule with a non-fluorescent acceptor dye attached to the substrate at either side of the substrate bond to be cleaved. A conjugate is prepared, the conjugate comprising a carrier peptide according to the invention, linked to the fluorogenic substrate component and as follows: Cy3-Gly-Ser-Gly-Asp-Glu-Val-Asp-Gly-Ser-Gly-Lys(Cy5Q)-Ala-Pro-Thr-Arg-Val-Leu-Pro-Pro-Leu-Val-Ala-Gly-Gly-Lys-amide. When Cy3 is in close proximity with Cy5Q (a quencher), fluorescence emission is markedly reduced. However, when the above peptide sequence is cleaved by an intracellular enzyme, such as caspase 3, Cy3 is no longer in close proximity with the quencher and fluorescence is emitted which can be measured with standard fluorescence instrumentation. The conjugate may be synthesised by techniques well known to the skilled person, for example by means of solid phase peptide synthesis methods as described in “Solid Phase Peptide Synthesis”, E. Atherton and R. C. Sheppard, IRL Press 1989. Labelling of the substrate component may be carried out using orthogonal protection strategies by coupling Cy3 mono acid (Amersham Pharmacia Biotech) to H₂N-Gly-Ser-Gly-Asp-Glu-Val-Asp-Gly-Ser-Gly-Lys-(Mtt)-Ala-Pro-Thr-Arg-Val-Leu-Pro-Pro-Leu-Val-Ala-Gly-Gly-Lys-(Boc)-Rink amide resin via in-situ activation, using 7-azabenzotriazol-1-yloxytris(pyrrolidino)phosphonium-hexafluorophosphate (PyAOP), 1-hydroxybenzotriazole (HOAt) and diisopropylamine in N-methylpyrrolidone (NMP). Removal of the methyltrityl (Mtt) protecting group, followed by coupling Cy5Q NHS ester to the deprotected peptide will yield the conjugate.

An assay for caspase 3 activity may be performed as follows. Cells are seeded into 96-well tissue culture plate and the conjugate containing the caspase substrate, Cy3-Gly-Ser-Gly-Asp-Glu-Val-Asp-Gly-Ser-Gly-Lys (Cy5Q)-Ala-Pro-Thr-Arg-Val-Leu-Pro-Pro-Leu-Val-Ala-Gly-Gly-Lys-amide is added to the cells at a final concentration of 10 μM for 60 minutes (5% CO₂, 37° C. 95% humidity). Apoptosis, and thus caspase enzyme activity is induced using agents such as 1 μM staurosporine, in the presence of suitable controls. Fluorescence, and therefore enzyme activity may be monitored with standard fluorescence instrumentation (activating at 530 nm, emission at 590 nm) over selected time points from 1 to 24 hours. The increase in fluorescence activity detected is an index of intracellular enzyme activity.

The invention is further illustrated by reference to the following examples and figures.

### FIGURES

FIG. 1 illustrates the dose-dependent uptake of Cy3 linked to carrier peptide: H₂N-Ala-Pro-Thr-Arg-Val-Leu-Pro-Pro-Leu-Val-Ala-Gly-Gly-Lys-OH) (SEQ ID No.6) into NIH 3T3 cells, according to Example 3. The data is shown as the mean ±1 standard deviation of three separate determinations.

FIG. 2a) (i) is a confocal laser scanning microscopy image of NIH 3T3 cells treated with Cy3 linked to carrier peptide: H₂N-Ala-Pro-Thr-Arg-Val-Leu-Pro-Pro-Leu-Val-Ala-Gly-Gly-Lys-OH (SEQ ID No.6); (ii) Unlinked Cy3 control, according to Example 3.

FIG. 2b) (i) is a confocal laser scanning microscopy image of SK-OV NTR cells treated with Cy3 linked to carrier peptide: H₂N-Ala-Pro-Thr-Arg-Val-Leu-Pro-Pro-Leu-Val-Ala-Gly-Gly-Lys-OH (SEQ ID No.6); (ii) Unlinked Cy3 control, according to Example 3.

FIG. 3 is a confocal laser scanning microscopy image of NIH 3T3 cells treated with Cy3 linked to carrier peptide: H₂N-Ala-Pro-Thr-Arg-Val-Leu-Pro-Pro-Leu-Val-Ala-Gly-Gly-Lys-OH (SEQ ID No.6), according to Example 3. Section 1, cellular base; Section 11, central region of the cells; Section 20; top of cells.

FIG. 4. NIH 3T3 cells were incubated with Cy3 linked to carrier peptides (H₂N-Ala-Pro-Thr-Arg-Val-Pro-Leu-Pro-Leu-Pro-Val-Gly-Gly-Lys-OH (SEQ ID No.4); H₂N-Ala-Pro-Thr-Arg-Ala-Val-Leu-Pro-Leu-Ala-Val-Gly-Gly-Lys-OH (SEQ ID No.5); H₂N-Ala-Pro-Thr-Arg-Val-Leu-Pro-Pro-Leu-Val-Ala-Gly-Gly-Lys-OH (SEQ ID No.6); H₂N-Ala-Pro-Thr-Arg-Val-Leu-Leu-Pro-Leu-Leu-Val-Gly-Gly-Lys-OH (SEQ ID No.7); H₂N-Ala-Pro-Thr-Arg-Leu-Pro-Leu-Pro-Leu-Val-Ala-Gly-Gly-Lys-OH (SEQ ID No.8), and cells examined by confocal microscopy, according to Example 3. The results were digitally recorded, and fluorescence data was further analyzed using a MetaMorph Image Processing software package. These data demonstrated up to a 10-fold increase in uptake of dye compared with the controls. The data is representative of three different experiments.

FIG. 5. NIH 3T3 cells were incubated with Cy3 linked to carrier peptide, H₂N-Ala-Pro-Thr-Arg-Val-Leu-Pro-Pro-Leu-Val-Ala-Gly-Gly-Lys-OH (SEQ ID No.6), and examined by flow cytometry, according to Example 4. Untreated cells and Cy3 only (unlinked dye) were used as controls. Each dot on the dot plots represents a single cell and the dot plots show a homogeneous uptake of Cy3 by NIH 3T3 cells.

FIG. 6. NIH 3T3 cells were incubated with Cy3 linked to carrier peptide, H₂N-Ala-Pro-Thr-Arg-Val-Leu-Pro-Pro-Leu-Val-Ala-Gly-Gly-Lys-OH (SEQ ID No.6), and examined by flow cytometry, according to Example 4. Untreated cells and Cy3 only (unlinked dye) were used as controls. The histogram plots show that there is an approximately 5-fold increase in the fluorescence geometric men between dye control and test cells treated with carrier peptide linked to Cy3.

FIG. 7. NIH 3T3 cells were incubated with Cy3 linked to carrier peptide, H₂N-Ala-Pro-Thr-Arg-Val-Leu-Pro-Pro-Leu-Val-Ala-Gly-Gly-Lys-OH (SEQ ID No.6), and examined by flow cytometry, according to Example 4. Cy3 only (unlinked dye) was used as a control. These data demonstrate that approximately 80% of cells are Cy3 labeled above that of the dye control.

FIG. 8. NIH 3T3 cells were incubated with Cy3-labelled carrier peptides (H₂N-Ala-Pro-Thr-Arg-Val-Pro-Leu-Pro-Leu-Pro-Val-Gly-Gly-Lys-OH (SEQ ID No.4); H₂N-Ala-Pro-Thr-Arg-Ala-Val-Leu-Pro-Leu-Ala-Val-Gly-Gly-Lys-OH (SEQ ID No.5); H₂N-Ala-Pro-Thr-Arg-Val-Leu-Pro-Pro-Leu-Val-Ala-Gly-Gly-Lys-OH (SEQ ID No.6); H₂N-Ala-Pro-Thr-Arg-Val-Leu-Leu-Pro-Leu-Leu-Val-Gly-Gly-Lys-OH (SEQ ID No.7), and subjected to differential permeabilsation, according to Example 5. Results indicate a significant amount of delivered agent in the cytosol of the cultured cells (a) the cellular pellet (organellor fraction) from cells treated with digitonin, (b) the supernatant (cytosolic fraction) from cells treated with digitonin, (c) the pellet from unpermeabilised cells, (d) the supernatant from unpermeabilised cells. The data is shown as the mean ±1 standard deviation of three separate determinations.

FIG. 9. NIH 3T3 cells were incubated with Cy5Q-labelled carrier peptide (H₂N-Ala-Pro-Thr-Arg-Val-Leu-Pro-Pro-Leu-Val-Ala-Gly-Gly-Lys-OH) (SEQ ID No.6), according to Example 6.2. The data shows a significant cellular uptake and substrate conversion with the delivery peptide linked to Cy5Q in a cell-based assay for nitroreductase. The data is representative of three separate experiments: (a) Parental SK-OV cells treated with ethyl ester linked Cy5Q, (b) Transfected SK-OV NTR cells treated with ethyl ester Cy5Q, (c) Parental SK-OV cells treated with carrier peptide linked to Cy5Q, (d) Transfected SK-OV NTR cells treated with carrier peptide linked to Cy5Q, (e) Transfected SK-OV NTR cells treated with unlinked Cy5Q.

FIG. 10. NIH 3T3 cells were cultured with carrier peptide (SEQ ID No.6) linked to Cy3-conjugated nuclear localization sequence to form: Cy3-Ser-Ser-Asp-Asp-Glu-Ala-Thr-Ala-Ser-Asp-Gln-His-Ser-Thr-Pro-Pro-Lys-Lys-Lys-Arg-Lys-Val-Glu-Asp-Pro-Lys-Ala-Pro-Thr-Arg-Val-Leu-Pro-Pro-Leu-Val-Ala-Gly-Gly-Lys-amide, according to Example 7.3. Controls included Cy3 linked to carrier peptide (Cy3-Ala-Pro-Thr-Arg-Val-Leu-Pro-Pro-Leu-Val-Ala-Gly-Gly-Lys-OH) (Seq ID No. 6), and unconjugated Cy3. Results indicated a more pronounced nuclear staining, (compared to the controls) with those cells treated with the carrier peptide linked to the nuclear localization sequence. (a) Cy3 conjugated to nuclear localisation sequence linked to carrier peptide (SEQ ID No.6); (b) Cy3 linked to carrier peptide (SEQ ID No.6); (c) unlinked Cy3.

FIG. 11 illustrates the dose-dependent uptake of GST linked to carrier peptide: H₂N-Thr-Lys-Lys-Pro-Leu-Pro-Pro-Thr-Pro-Glu-Glu-Asp-OH (SEQ ID No.1) into NIH 3T3 cells, according to Example 8.2. The data is shown as the mean ±1 standard deviation of three separate determinations.

FIG. 12 (A) is an immunofluorescence image, captured on a Nikon-Diaphot 300 microscope, according to Example 8.2, of 3T3 cells treated with GST-linked to carrier peptide: H₂N-Thr-Lys-Lys-Pro-Leu-Pro-Pro-Thr-Pro-Glu-Glu-Asp-OH (SEQ ID No.1); (B) GST wild type control.

FIG. 13 (A) is a confocal laser scanning microscopy image, according to Example 8.2, of NIH 3T3 cells treated with GST linked to carrier peptide: H₂N-Thr-Lys-Lys-Pro-Leu-Pro-Pro-Thr-Pro-Glu-Glu-Asp-OH (SEQ ID No.1); (B) GST wild type control.

### EXAMPLES

1. Chemical Synthesis of Carrier Peptides

The following carrier peptides were synthesized using a commercially available Perkin-Elmer Model 431A automated peptide synthesizer and FastMoc™ chemistry, following the instrument manufacturer's recommended procedures throughout:

| | |
|---|---|
| i) | (SEQ ID NO:1) |

H₂N-Thr-Lys-Lys-Pro-Leu-Pro-Pro-Thr-Pro-Glu-Glu-
ASP-OH;

| | |
|---|---|
| ii) | (SEQ ID NO:2) |

H₂N-Ser-Glu-Pro-Ala-Val-Ser-Pro-Leu-Leu-Pro-Arg-
Lys-Glu-Arg-OH;

| | |
|---|---|
| iii) | (SEQ ID NO:3) |

H₂N-Ala-Pro-Thr-Met-Pro-Pro-Pro-Leu-Pro-Pro-Leu-
Gly-Gly-Lys-OH;

| | |
|---|---|
| iv) | (SEQ ID NO:4) |

H₂N-Ala-Pro-Thr-Arg-Val-Pro-Leu-Pro-Leu-Pro-Val-
Gly-Gly-Lys-OH;

| | |
|---|---|
| v) | (SEQ ID NO:5) |

H₂N-Ala-Pro-Thr-Arg-Ala-Val-Leu-Pro-Leu-Ala-Val-
Gly-Gly-Lys-OH;

| | |
|---|---|
| vi) | (SEQ ID NO:6) |

H₂N-Ala-Pro-Thr-Arg-Val-Leu-Pro-Pro-Leu-Val-Ala-
Gly-Gly-Lys-OH;

| | |
|---|---|
| vii) | (SEQ ID NO:7) |

H₂N-Ala-Pro-Thr-Arg-Val-Leu-Leu-Pro-Leu-Leu-Val-
Gly-Gly-Lys-OH;

| | |
|---|---|
| viii) | (SEQ ID NO:8) |

H₂N-Ala-Pro-Thr-Arg-Leu-Pro-Leu-Pro-Leu-Val-Ala-
Gly-Gly-Lys-OH.

Syntheses were performed on a 0.25 millimolar scale and, on completion, the peptides were cleaved from the solid phase using standard trifluoroacetic acid procedures. The crude peptides obtained from the cleavage reactions were purified by conventional C-18 reverse phase HPLC using a linear gradient of water/acetonitrile (both containing 0.1% trifluoroacetic acid). After purification, the peptides were lyophilized to give colourless solids. The molecular weights of the purified peptides were verified by Maldi Tof mass spectrometry and, the amino acid compositions confirmed using amino acid analysis.

2. Conjugation to Cy3

Carrier peptides (H₂N-Thr-Lys-Lys-Pro-Leu-Pro-Pro-Thr-Pro-Glu-Glu-Asp-OH (SEQ ID No.1); H₂N-Ser-Glu-Pro-Ala-Val-Ser-Pro-Leu-Leu-Pro-Arg-Lys-Glu-Arg-OH (SEQ ID No.2); H₂N-Ala-Pro-Thr-Met-Pro-Pro-Pro-Leu-Pro-Pro-Leu-Gly-Gly-Lys-OH (SEQ ID No.3); H₂N-Ala-Pro-Thr-Arg-Val-Pro-Leu-Pro-Leu-Pro-Val-Gly-Gly-Lys-OH (SEQ ID No.4); H₂N-Ala-Pro-Thr-Arg-Ala-Val-Leu-Pro-Leu-Ala-Val-Gly-Gly-Lys-OH (SEQ ID No.5); H₂N-Ala-Pro-Thr-Arg-Val-Leu-Pro-Pro-Leu-Val-Ala-Gly-Gly-Lys-OH (SEQ ID No.6); H₂N-Ala-Pro-Thr-Arg-Val-Leu-Leu-Pro-Leu-Leu-Val-Gly-Gly-Lys-OH (SEQ ID No.7); H₂N-Ala-Pro-Thr-Arg-Leu-Pro-Leu-Pro-Leu-Val-Ala-Gly-Gly-Lys-OH (SEQ ID No.8), were linked to Cy3 through an activated N-hydroxysuccinimide ester in the presence of dry dimethylsulfoxide and N,N-diisopropylethylamine. (Erlanger, B. F. Principles and methods for the preparation of drug protein conjugates for immunological studies, Pharmacol. Rev., 1973, (25), 271-280). The peptides were linked to dye at molar ratios of 1 mole of peptide to 1.1 mole of dye. The reaction mixture was incubated overnight at room temperature.

The labelled peptides were purified by conventional C-18 reverse phase HPLC using a linear gradient of water/acetonitrile (both containing 0.1% trifluoroacetic acid). After purification, the peptides were lyophilized. The molecular weights of the labelled peptides were verified by Maldi Tof mass spectrometry.

3. Cellular Uptake of Cy3

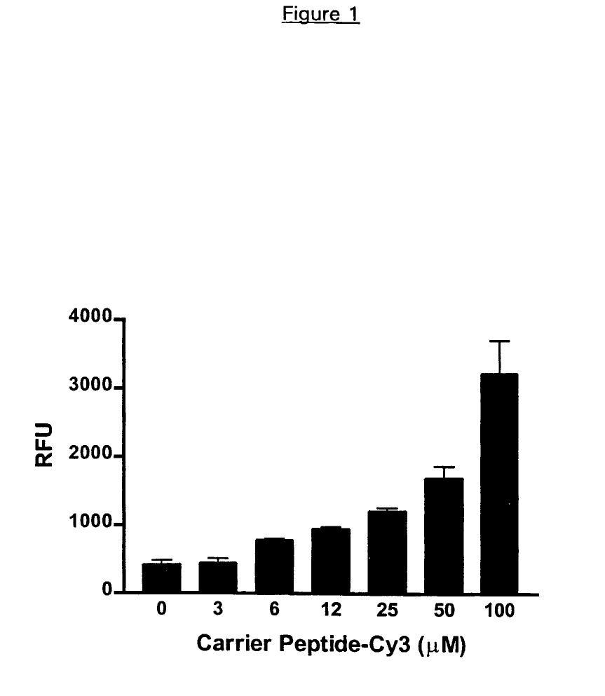

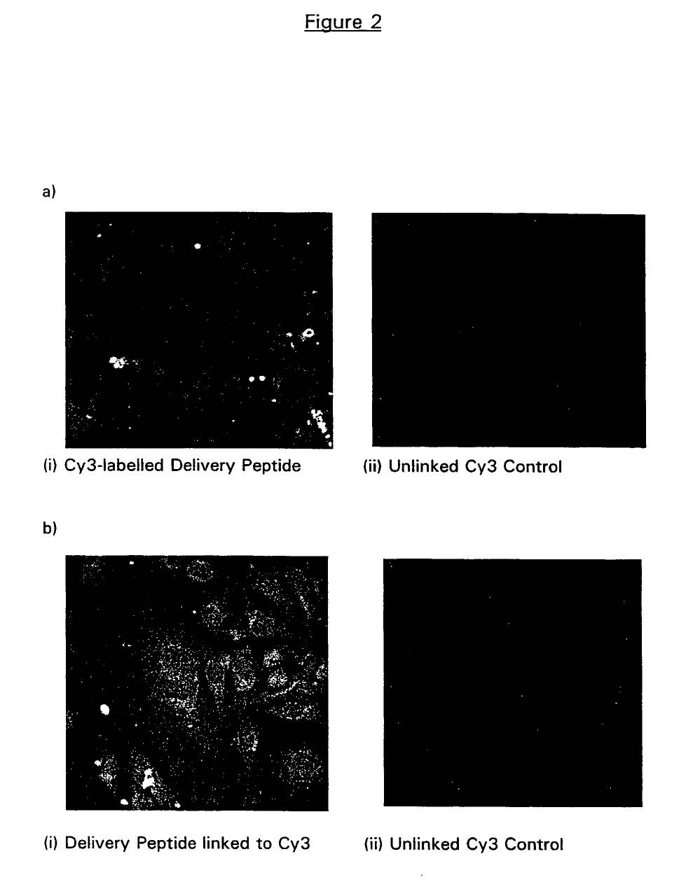

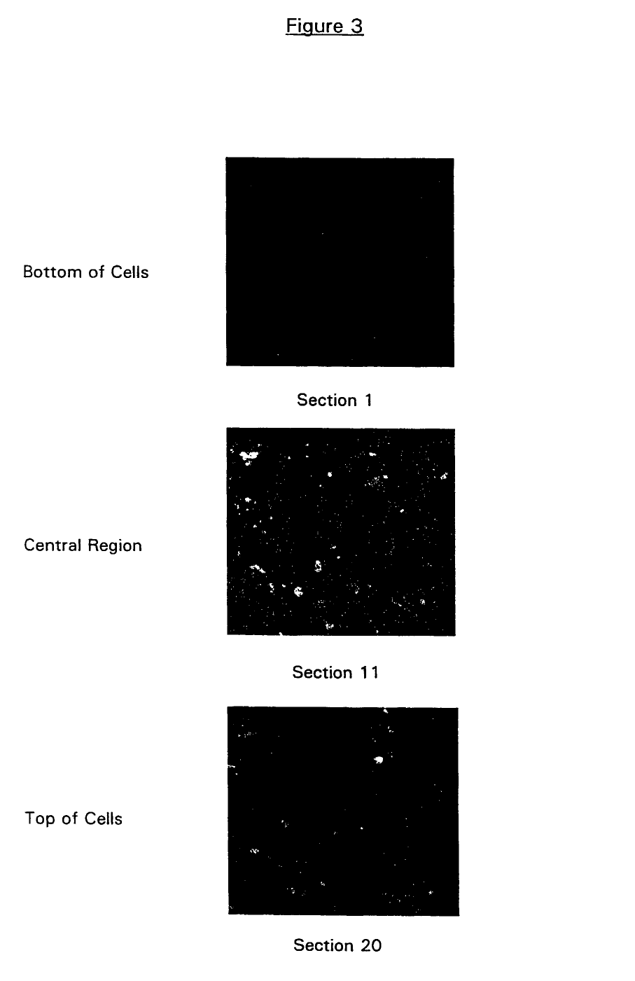

Mouse fibroblasts (NIH 3T3) or human Caucasian ovary adenocarcinoma (SK-OV) cells were cultured in Dulbecco's modified Eagle's medium (DMEM) supplemented with 10% (v/v) foetal calf serum (FCS), 4 mM L-glutamine, 100 U/ml penicillin and 100 μg/ml streptomycin. NIH 3T3 cells were grown on 96-well tissue culture plates (Costar Inc.) or WillCo-dishes (glass-bottomed dishes, with 0.17 mm cover glass for inverted microscopes) (WillCo), before treatment with carrier peptide. Cells were incubated (60 min, 5% CO₂, 37° C., 95% humidity) with Cy3 linked to carrier peptide (H₂N-Ala-Pro-Thr-Arg-Val-Leu-Pro-Pro-Leu-Val-Ala-Gly-Gly-Lys-OH) (SEQ ID No.6)) (range 1-100 μM). After incubation, the culture supernatant was decanted and the cells were washed. Cy3 (unlinked dye) (Amersham Pharmacia Biotech) was used as a control. Cellular fluorescence was detected using a CytoFluor multi-well plate reader (Applied Biosystems, Foster City, Calif., USA), (activating at 530 nm, emission 590 nm) (FIG. 1); a Nikon-Diaphot 300 fluorescence microscope, (activating at 490 nm, emission at 520 nm); and a Zeiss Microsystems confocal laser scanning microscope (LSM4) (activating at 515 nm, emission at 565 nm) (FIGS. 2 (a) and (b)). Cells were further studied for intracellular dye localization by a twenty-step Z-position sectional scanning of the cell (0.5 μm/section) using a 40× oil immersion lens (FIG. 3). The images clearly show dye staining in the central portion of the cell, and little staining on the periphery or cell membrane.

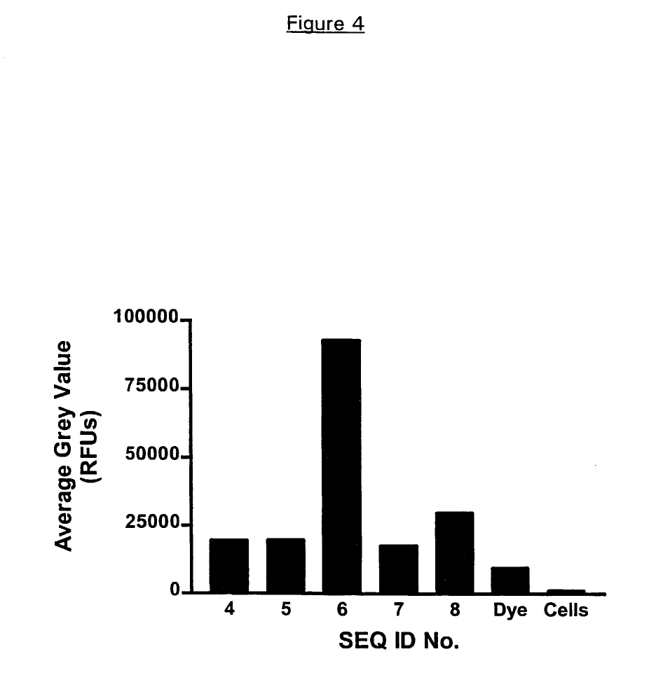

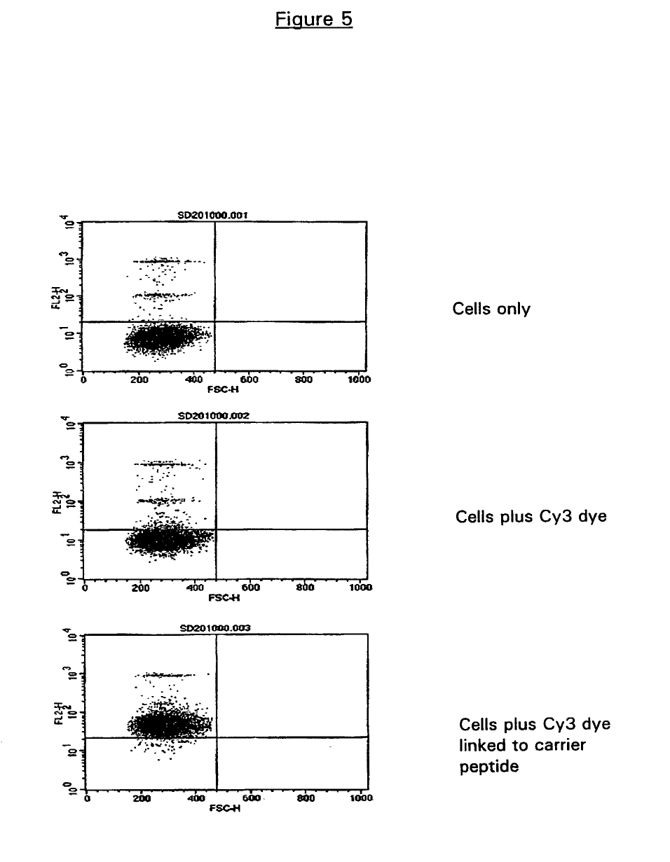

NIH 3T3 cells were incubated with Cy3 linked to carrier peptides (H₂N-Ala-Pro-Thr-Arg-Val-Pro-Leu-Pro-Leu-Pro-Val-Gly-Gly-Lys-OH (SEQ ID No.4); H₂N-Ala-Pro-Thr-Arg-Ala-Val-Leu-Pro-Leu-Ala-Val-Gly-Gly-Lys-OH (SEQ ID No.5); H₂N-Ala-Pro-Thr-Arg-Val-Leu-Pro-Pro-Leu-Val-Ala-Gly-Gly-Lys-OH (SEQ ID No.6); H₂N-Ala-Pro-Thr-Arg-Val-Leu-Leu-Pro-Leu-Leu-Val-Gly-Gly-Lys-OH (SEQ ID No.7); H₂N-Ala-Pro-Thr-Arg-Leu-Pro-Leu-Pro-Leu-Val-Ala-Gly-Gly-Lys-OH (SEQ ID No.8)), and the cells examined by confocal microscopy. The data was recorded digitally and fluorescence data was further analyzed with the MetaMorph Image Processing software package (Universal Imaging Corporation, West Chester, Pa., USA). This demonstrated a 10-fold increase in uptake of dye compared with the controls (FIG. 4).

NIH 3T3 cells were cultured on WillCo-dishes (glass-bottomed dishes, with 0.17 mm cover glass for inverted microscopes) (WillCo), before treatment with carrier peptides linked to Cy3. Confluent NIH 3T3 cells were incubated (60 min, 5% CO₂, 37° C., 95% humidity) with 1,1′-dioctadecyl-3,3,3′,3′-tetramethylindodicarbocyanine-5,5′-disulfonic acid (DilC18 (5)-DS) (Dil) (Molecular Probes, D-12730) (20 μM) (a cell membrane stain) and carrier peptide linked to Cy3 (10 μM). Similarly, cells were incubated (as above) with Syto61 red fluorescent nucleic acid stain (5 μM) and carrier peptide linked to Cy3. The cells were washed (×3) with PBS, fixed with 80% (v/v) ethanol before visualization using a Zeiss Microsystems confocal laser-scanning microscope. The appropriate excitation and emission spectra were Cy3 (515/565 nm), Dil (650/670 nm) and Syto61 (620/647 nm). Data (not shown) clearly demonstrated internalization of the Cy3 dye linked to the carrier peptide as indicated by the membrane stain Dil. The majority of Cy3 staining appeared to be cytosolic, with less uptake of dye in the nuclear region of the cell (indicated by the nuclear stain Syto61).

4. Flow Cytometry

Mouse fibroblasts (NIH 3T3 cells) were cultured in DMEM (see above) on 24-well tissue culture plates (Costar Inc.). Cells were incubated (60 min, 5% CO₂, 37° C., 95% humidity) with Cy3 (1 μM) linked to carrier peptide (H₂N-Ala-Pro-Thr-Arg-Val-Leu-Pro-Pro-Leu-Val-Ala-Gly-Gly-Lys-OH (SEQ ID No.6)). After incubation, the culture supernatant was decanted and the cells washed. Cells were trypsinised (0.25% w/v, 5 min) and re-suspended in PBS, before analysis by flow cytometry (see below). Untreated cells and Cy3 only (unlinked dye) (Amersham Pharmacia Biotech) were used as controls.

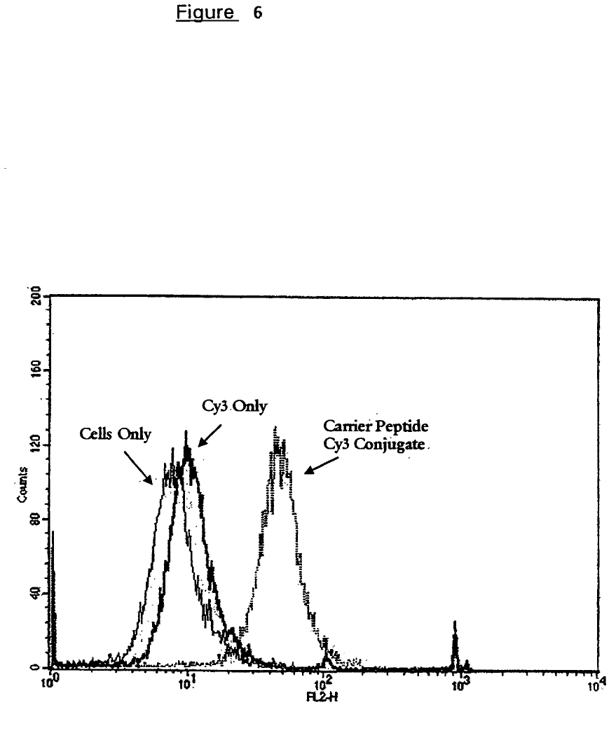

All samples were analyzed on a FACSCalibur Flow Cytometry System (Becton Dickinson, San Jose, Calif., USA) with CellQuest software. The instrument is equipped with a 488 nm argon-ion laser and 5 detection parameters. There are 2 scatter detectors, known as forward scatter (FSC) and side scatter (SSC) which provide information about cellular size and internal complexity, respectively. In addition, there are three relative fluorescence intensity detectors. Cy3 labelling was monitored with the second fluorescence detector (FL2) fitted with a 585/42 nm filter. 10,000 cells were collected for analysis on a log scale and a “gate” drawn around cells with typical forward and side scatter characteristics for viable cells. Each dot on the dot-plots represents a single cell and the dot plots show a homogeneous uptake of Cy3 by NIH 3T3 cells (FIG. 5). The flow cytometry histogram plots show that there is an approximately 5-fold increase in the fluorescence geometric mean between dye control and test cells (FIG. 6).

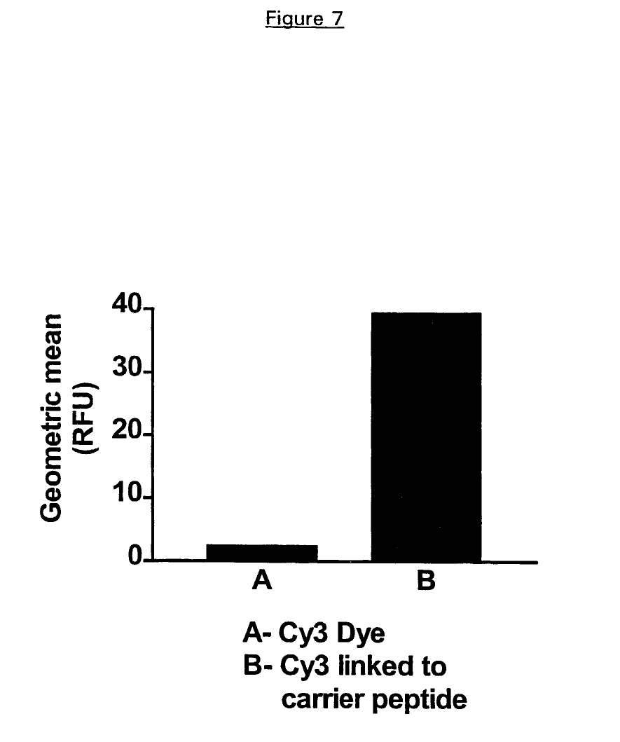

Histogram markers were set so that only 2-3% of the recorded events were past the FL2 channel marker 30 for the dye only control. The same analysis marker was then applied to the test sample, demonstrating that approximately 88% of cells are Cy3 labelled above that of the dye control (FIG. 7).

5. Selective Permeabilisation with Digitonin

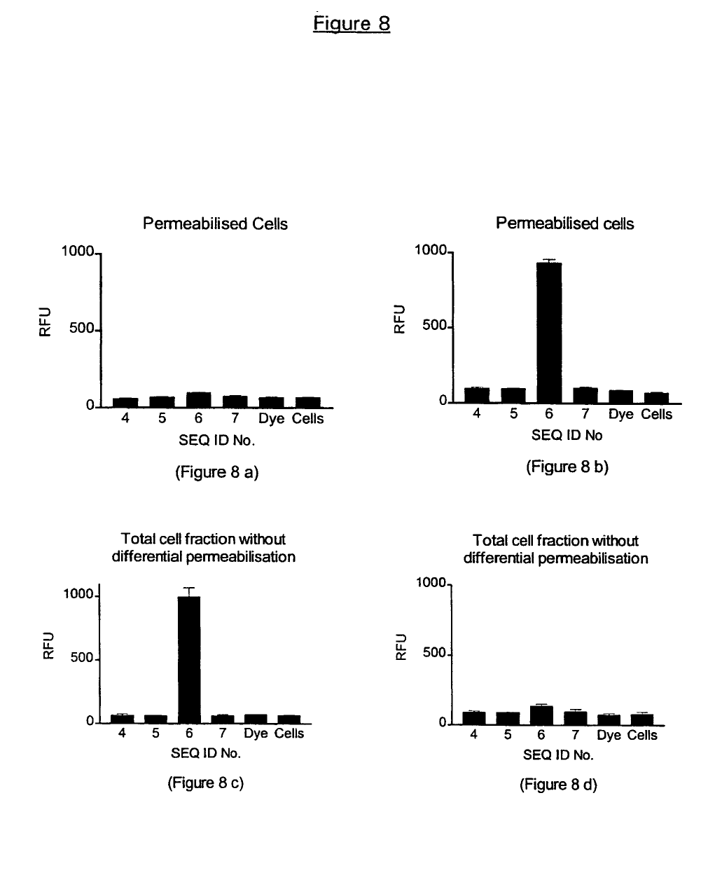

Mouse fibroblasts (NIH 3T3) were cultured at 1×10⁶ cells/ml in DMEM (see above) on 24-well tissue culture plates (Costar Inc.). Cells were incubated with Cy3- labelled carrier peptides (H₂N-Ala-Pro-Thr-Arg-Val-Pro-Leu-Pro-Leu-Pro-Val-Gly-Gly-Lys-OH (SEQ ID No.4); H₂N-Ala-Pro-Thr-Arg-Ala-Val-Leu-Pro-Leu-Ala-Val-Gly-Gly-Lys-OH (SEQ ID No.5); H₂N-Ala-Pro-Thr-Arg-Val-Leu-Pro-Pro-Leu-Val-Ala-Gly-Gly-Lys-OH (SEQ ID No.6); H₂N-Ala-Pro-Thr-Arg-Val-Leu-Leu-Pro-Leu-Leu-Val-Gly-Gly-Lys-OH (SEQ ID No.7) for 60 minutes (5% CO₂, 37° C., 95% humidity). Cells were washed (×4) with PBS, before addition of 20 μM digitonin for 3 minutes. Cells were rapidly centrifuged (10,000×g, for 5 minutes) to separate into supernatant (cytosolic) and pellet (organelle) fractions. The fluorescence activity was then measured using a CytoFluor multi-well plate reader (activating at 530 nm, emission 590 nm). Unlabelled cells (cells without dye), unlabelled Cy3 (unlinked dye) (Amersham Pharmacia Biotech), and, untreated cells (unpermeabilised cells without digitonin), were used as controls. Results are shown in FIG. 8, indicating a significant amount of delivered dye into the cytosol of the cultured cells, in particular using H₂N-Ala-Pro-Thr-Arg-Val-Leu-Pro-Pro-Leu-Val-Ala-Gly-Gly-Lys-OH (SEQ ID No.6). Cytosolic preparations were confirmed using an assay for cytoplasmic lactic dehydrogenase (LDH) in the presence of PNADH (Wroblewski & LaDue, Proc. Soc. Exp. Biol. Med. 1955, (90), 210-213.

6. Nitroreductase Assay

As disclosed in PCT/GB99/01746, the fluorescence properties of cyanine dyes may be modified by substitution of the dye with nitro, and/or dinitrobenzyl groups, which have the effect of reducing or abolishing the fluorescence from the molecule. An example of such a quenching or “dark” dye is Cy5Q (Amersham Pharmacia Biotech). In the presence of the enzyme, nitroreductase (NTR), a nitro-group substituent of Cy5Q may be converted to an amino-group, with the result that there is an increase in the fluorescence intensity of the dye. This may be used as the basis for an assay for the measurement of intracellular nitroreductase. In this example, Cy5Q is conjugated to a carrier peptide and is used as an indicator of nitroreductase (NTR) in whole, living cells.

The assay may be carried out using cells transfected with DNA encoding the gene for bacterial NTR, such that when the Cy5Q conjugate is introduced into such a transfected cell line, an increase in fluorescence is measured.

6.1 Synthesis of Ac-Ala-Pro-Thr-Arg-Val-Leu-Pro-Pro-Leu-Val-Ala-Gly-Gly-Lys(Cy5Q)-amide (Nitroreductase Reporter)

(i) Ac-Ala-Pro-Thr-Arg-Val-Leu-Pro-Pro-Leu-Val-Ala-Gly-Gly-Lys-Rink Amide Resin

Ac-Ala-Pro-Thr-Arg-Val-Leu-Pro-Pro-Leu-Val-Ala-Gly-Gly-Lys-Rink amide resin was synthesized using a commercially available Applied Biosystems Model 433A automated peptide synthesizer and FastMoc™ chemistry, following the instrument manufacturer's recommended procedures throughout. The synthesis was performed on a 0.25 millimolar scale. The resin was removed from the peptide synthesizer and dried in vacuo.

(ii) Synthesis of Ac-Ala-Pro-Thr-Arg-Val-Leu-Pro-Pro-Leu-Val-Ala-Gly-Gly-Lys-amide

Crude peptide was deprotected and cleaved from the solid phase using a mixture of 95% trifluoroacetic acid: 2.5% water: 2.5% triisopropylsilane. The crude peptide obtained from the cleavage reaction was purified by conventional C-18 reverse phase HPLC using a linear gradient of water/acetonitrile (both containing 0.1% trifluoroacetic acid). After purification, the peptide was lyophilized and characterized by Maldi Tof mass spectroscopy and HPLC.

(iii) Synthesis of Ac-Ala-Pro-Thr-Arg-Val-Leu-Pro-Pro-Leu-Val-Ala-Gly-Gly-Lys(Cy5Q)-amide

Ac-Ala-Pro-Thr-Arg-Val-Leu-Pro-Pro-Leu-Val-Ala-Gly-Gly-Lys-amide was labelled at the C-terminal in solution phase, with Cy5Q NHS ester (1.5 eq) (Amersham Pharmacia Biotech) in dimethylsulfoxide and diisopropylethylamine (4% v/v). The reaction mixture was purified by C-18 reverse phase HPLC using a linear gradient of water/acetonitrile (both containing 0.1% trifluoroacetic acid). After purification, the mono-labelled peptide was lyophilised and characterized by Maldi Tof mass spectroscopy, UV and HPLC.

6.2 Delivery of Nitroreductase Reporter

Human Caucasian ovary adenocarcinoma (SK-OV-3) (ECACC ref No. 91091004) cells were stably transfected with bacterial NTR genes. These aforementioned transfected cells are now subsequently referred to as SK-OV-NTR cells. Non-transfected cells are referred to as parental SK-OV cells. All experiments were carried out in black, 96-well tissue culture plates with clear bases.

Cy5Q chemically linked to the carrier peptide Ac-Ala-Pro-Thr-Arg-Val-Leu-Pro-Pro-Leu-Val-Ala-Gly-Gly-Lys-amide was incubated with cells (30,000 cells/well) (1-5 hours, 5% CO₂, 37° C., 95% humidity), cells washed (×3) with PBS and fluorescence measured from below, using a CytoFluor multi-well plate reader (Applied Biosystems), (activating at 610 nm, emission 670 nm). Controls in these experiments included:

a) Parental SK-OV cells treated with ethyl ester Cy5Q (negative control);

b) SK-OV NTR cells incubated with ethyl ester linked Cy5Q (positive control);

c) Parental SK-OV cells treated with the delivery peptide linked to Cy5Q (negative control); and

d) SK-OV NTR cells incubated with unlabelled Cy5Q (negative control).

All test and control reagents were used at a final concentration of 10 μM.

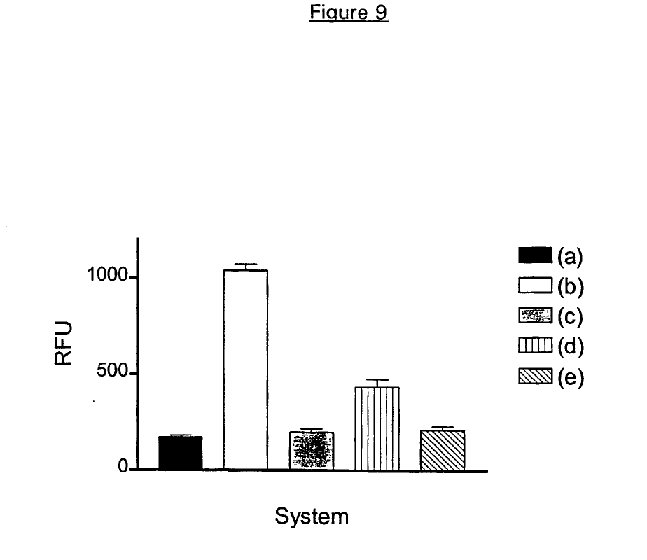

The data (FIG. 9) shows a profound uptake and conversion of the ethyl ester linked Cy5Q after a 5 hour incubation with the transfected cells. However, the data also demonstrates a significant cellular internalization and substrate conversion with the carrier peptide linked to Cy5Q. Little conversion was observed with Cy5Q delivered, using either peptide linked-, or ethyl ester linked Cy5Q, to the parental cell lines. Unconjugated Cy5Q was not delivered to the SK-OV NTR cells. The results are representative of three separate experiments.

These data clearly indicates the utility of the carrier peptide in a whole cell assay approach, and also demonstrates a significant uptake of the peptide into the cytosol of the transfected SK-OV NTR cells.

7.

Delivery of Nuclear Localization Sequence:
Cy3-Ser-Ser-Asp-Asp-Glu-Ala-Thr-Ala-Ser-Asp-Gln-
His-Ser-Thr-Pro-Pro-Lys-Lys-Lys-Arg-Lys-Val-Glu-
Asp-Pro-Lys-Ala-Pro-Thr-Arg-Val-Leu-Pro-Pro-Leu-
Val-Ala-Gly-Gly-Lys-amide

Particular peptide sequences have the ability to localize to unique regions within the cell. This capacity may be utilized in combination with the carrier peptide to deliver a cyanine dye to a pre-defined region of the cell. The example given here is a nuclear localization sequence described in WO 99/07723 and which binds to nuclear material. Thus, a cyanine dye is targeted to nucleus of a live cell.

7.1

Synthesis of H2N-Ser-Ser-Asp-Asp-Glu-Ala-Thr-Ala-
Ser-Asp-Gln-His-Ser-Thr-Pro-Pro-Lys-Lys-Lys-Arg-
Lys-Val-Glu-Asp-Pro-Lys-Ala-Pro-Thr-Arg-Val-Leu-
Pro-Pro-Leu-Val-Ala-Gly-Gly-Lys-Rink Amide resin

H₂N-Ser-Ser-Asp-Asp-Glu-Ala-Thr-Ala-Ser-Asp-Gln-His-Ser-Thr-Pro-Pro-Lys-Lys-Lys-Arg-Lys-Val-Glu-Asp-Pro-Lys-Ala-Pro-Thr-Arg-Val-Leu-Pro-Pro-Leu-Val-Ala-Gly-Gly-Lys-Rink Amide resin was synthesized using a commercially available Applied Biosystems Model 433A automated peptide synthesizer and FastMoc™ chemistry, following the instrument manufacturer's recommended procedures throughout. The synthesis was performed on a 0.1-millimolar scale. The resin was removed from the peptide synthesizer and dried in vacuo.

7.2

Synthesis of Cy3-Ser-Ser-Asp-Asn-Glu-Ala-Thr-Ala-
Ser-Asp-Gln-His-Ser-Thr-Pro-Pro-Lys-Lys-Lys-Arg-
Lys-Val-Glu-Asp-Pro-Lys-Ala-Pro-Thr-Arg-Val-Leu-
Pro-Pro-Leu-Val-Ala-Gly-Gly-Lys-amide

Cy3 mono acid (1 eq) (Amersham Pharmacia Biotech) was coupled to H₂N-Ser-Ser-Asp-Asp-Glu-Ala-Thr-Ala-Ser-Asp-Gln-His-Ser-Thr-Pro-Pro-Lys-Lys-Lys-Arg-Lys-Val-Glu-Asp-Pro-Lys-Ala-Pro-Thr-Arg-Val-Leu-Pro-Pro-Leu-Val-Ala-Gly-Gly-Lys-Rink amide resin via in-situ activation, using 7-azabenzotriazol-1-yloxytris(pyrrolidino)-phosphonium-hexafluorophosphate (PyAOP) (1.5 eq), 1-hydroxy-7-azabenzotriazole (HOAt)(1.5 eq) and diisopropylethylamine (3 eq) in N-methyl pyrrolidone (NMP) at room temperature overnight. The reaction solvent was filtered off and the resin washed with N-methyl pyrrolidone*, dichloromethane and finally diethyl ether before drying in vacuo. *Unlabelled peptide can be capped at this point with a standard capping solution (0.5M acetic anhydride, 0.125M diisopropylethylamine, 0.015M hydroxybenzotriazole).

The crude peptide was cleaved from the solid phase using a mixture of 95% trifluoroacetic acid: 2.5% water: 2.5% triisopropylsilane. The crude peptide obtained from the cleavage reaction was purified by conventional C-18 reverse phase HPLC using a linear gradient of water/acetonitrile (both containing 0.1% trifluoroacetic acid). After purification, the peptide was lyophilized and characterized by Maldi Tof mass spectroscopy, UV and HPLC.

7.3

Cellular Delivery of Nuclear Localization
Sequence:
Cy3-Ser-Ser-Asp-Asp-Glu-Ala-Thr-Ala-Ser-Asp-Gln-
His-Ser-Thr-Pro-Pro-Lys-Lys-Lys-Arg-Lys-Val-Glu-
Asp-Pro-Lys-Ala-Pro-Thr-Arg-Val-Leu-Pro-Pro-Leu-
Val-Ala-Gly-Gly-Lys-amide

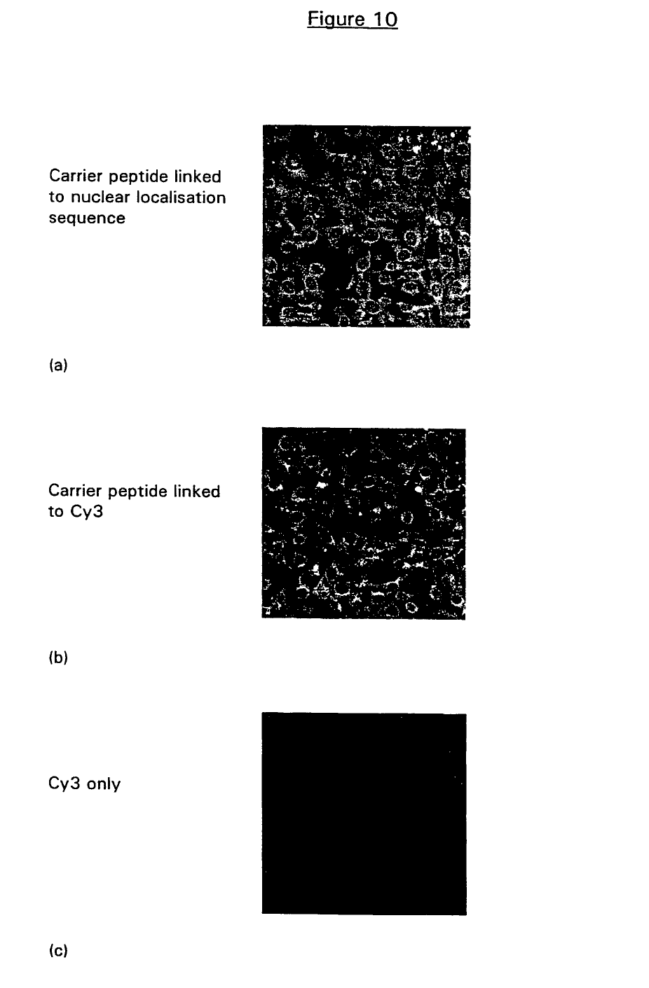

NIH 3T3 cells were cultured (see above) on WillCo-dishes (glass-bottomed dishes, with 0.17 mm cover glass for inverted microscopes) (WillCo), before treatment with carrier peptide linked to the nuclear localization sequence. Confluent NIH 3T3 cells were incubated (overnight, 5% CO₂, 37° C., 95% humidity) in the presence of Cy3-Ser-Ser-Asp-Asp-Glu-Ala-Thr-Ala-Ser-Asp-Gln-His-Ser-Thr-Pro-Pro-Lys-Lys-Lys-Arg-Lys-Val-Glu-Asp-Pro-Lys-Ala-Pro-Thr-Arg-Val-Leu-Pro-Pro-Leu-Val-Ala-Gly-Gly-Lys-amide, before washing (×3) with PBS, and visualization using a Zeiss Microsystems confocal laser scanning microscope (LSM4) (activating at 515 nm, emission at 565 nm). Controls included Cy3 linked to carrier peptide (Cy3-Ala-Pro-Thr-Arg-Val-Leu-Pro-Pro-Leu-Val-Ala-Gly-Gly-Lys-OH), and unlabelled Cy3 (unlinked dye) (Amersham Pharmacia Biotech). Results (FIG. 10) indicated a more pronounced nuclear staining with those cells treated with the carrier peptide linked to the nuclear localization sequence, as compared with the controls.

8. Delivery of the Protein Glutathione S-Transferase

8.1 Glutathione S-Transferase Conjugation

Carrier peptides (NH₂-Thr-Lys-Lys-Pro-Leu-Pro-Pro-Thr-Pro-Glu-Glu-Asp-OH; NH₂-Ser-Glu-Pro-Ala-Val-Ser-Pro-Leu-Leu-Pro-Arg-Lys-Glu-Arg-OH; NH₂-Ala-Pro-Thr-Met-Pro-Pro-Pro-Leu-Pro-Pro-Leu-Gly-Gly-Lys-OH) were coupled to glutathione S-transferase (GST) using hetero-bifunctional-coupling approaches (Aslam, M. & Dent, A. (Eds) Bioconjugation: Protein Coupling Techniques for the Biomedical Sciences, Macmillan Press (1998)). GST (EC 2.5.1.18; Sigma) was conjugated to each of the peptides using thiol and maleimide functional groups. Briefly, GST (4.86 mg; 1.92×10⁻⁷ moles) was desalted into PBS. S-Acetylthioglycolic acid N-hydroxysuccinimide ester (SATA; Sigma) (1.25 mg; 5.41×10⁻⁶ moles; 28:1 molar ratio) was dissolved in dry DMF (20 μl). SATA was added to GST and incubated for 1 hour at room temperature. The reaction was stopped and the thiol deprotected by adding the following: 0.1 M Tris/HCl, pH 7.0 (150 μl); 0.1M EDTA solution, pH 7.0 (30 μl); 1M hydroxylamine, pH 7.0 prepared in 0.1 M Tris/HCI, pH 7 (150 μl). (This reagent was prepared immediately before use.)

The reaction mixture was incubated at room temperature for 15 minutes. The sample was desalted on a Rapid Desalt Column delivered by FPLC™ (Amersham Pharmacia Biotech) and eluted with 10 μM phosphate buffer pH 6.0 containing 5 mM EDTA. The peptides (0.5 mg) (2.76×10⁻⁷ moles) were dissolved dry DMF or ethanol. 4-(N-Maleimidomethyl) cyclohexane-1-carboxylic acid N-hydroxysuccinimide ester (SMCC; Sigma) (1 mg) was dissolved in dry DMF (1 ml) and 100 μl (9.8×10⁻⁷ moles) (4:1 molar ratio) was added to the peptide solution. The reaction mixture was incubated for 1 hour at room temperature. The sample was applied to a Peptide Column™ eluting with 10 mM phosphate buffer pH 6.0 containing 5 mM EDTA, delivered by FPLC™. The first peak eluted contained the peptide incorporating a free maleimide moiety.

The thiolated GST and the activated peptide were combined at a molar ratio of 4:1 and incubated overnight at room temperature with constant mixing. The sample was concentrated in a Centriprep 10 (Amicon) and purified on Superdex 75™ (FPLC) eluting with water. The linked peptides were lyophilized. Conjugates were characterized using SDS polyacrylamide gel electrophoresis; size exclusion chromatography and GST enzyme activity detection kit (Amersham Pharmacia Biotech).

8.2 Cellular Uptake of Glutathione “S” Transferase (GST)

Mouse fibroblasts (NIH 3T3 cells) were cultured in Dulbecco's modified Eagle's medium (DMEM) supplemented with 10% (v/v) foetal calf serum (FCS), 4 mM L-glutamine, 100 U/ml penicillin, and 100 μg/ml streptomycin. NIH 3T3 cells were grown on 96-well tissue culture plates (Costar Inc.) or WillCo-dishes (glass-bottomed dishes, with 0.17 mm cover glass for inverted microscopes) (WillCo), before treatment with conjugates. Confluent NIH 3T3 cells were incubated (0.5-18 hours, 5% CO₂, 37° C., 95% humidity), with GST linked to carrier peptide at concentrations ranging from between 0.6-20 μM. After incubation, the culture supernatant was decanted and the cells washed. Optimal uptake occurred within 1 hour of incubation. GST-WT (unlinked protein) (Sigma) was always used a control. In a separate series of experiments GST (20 μM) uptake was tested at 4° C., 22° C. and 37° C. for 1 hour. Cells were further processed as described below.

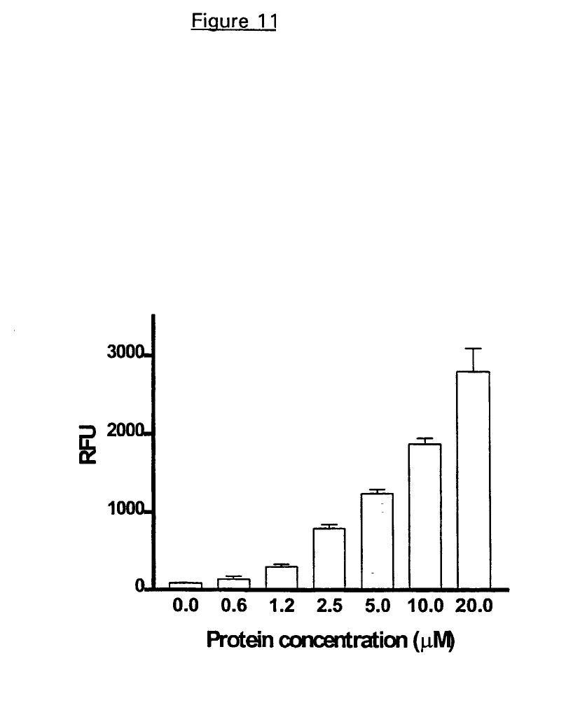

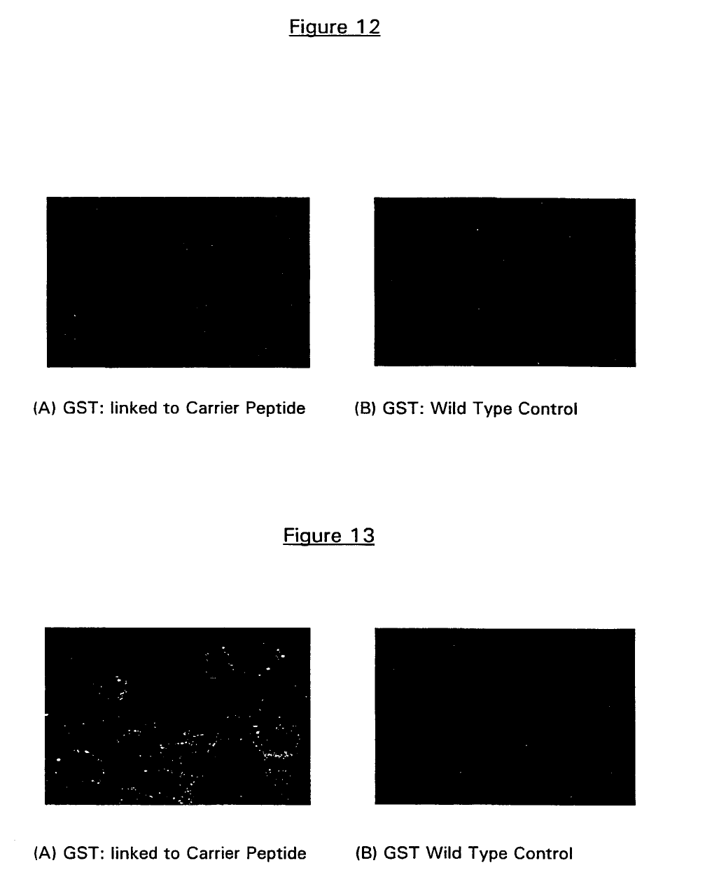

For immunofluorescence studies, washed (×3 with phosphate buffered saline) NIH 3T3 cells were fixed with 4% (v/v) paraformaldehyde for 10 minutes before treatment with 0.1% (w/v) saponin for 5 minutes. Cells were washed as above, and non-specific binding sites blocked with 3% (v/v) normal goat sera/1% bovine serum albumin (BSA) for 1 hour. The cells were washed, before incubating with rabbit anti-GST antibodies (Sigma), diluted at 1:1000 in PBS containing 1% (w/v) BSA for 1 hour. Cells were washed as before, and incubated with Cy3-labelled goat anti-rabbit Ig (Amersham Pharmacia Biotech), diluted at 1:1000 in PBS containing 1% (w/v) BSA for 1 hour. Cellular fluorescence was detected using a Biolumin 960 Kinetic Fluorescence Reader (Molecular Dynamics Corp.), (activating at 535 nm, emission 569 nm); a Nikon-Diaphot 300 fluorescence microscope, (activating at 490 nm, emission at 520 nm); and a Zeiss Microsystems confocal laser scanning microscope (LSM4) (activating at 515 nm, emission at 565 nm). Cells were further studied for intracellular protein localization by a ten-step Z-position sectional scanning of the cell (1 μm/section) using a 40× oil immersion lens. Positive assay controls consisted of untreated 3T3 cells stained with a monoclonal anti-fibronectin antibody (1:1000) (Sigma) and Cy-3 linked donkey anti-mouse Ig (Amersham Pharmacia Biotech). Negative assay controls consisted of a monoclonal antibody to murine interleukin-6 detected, cells without primary antibody but treated with Cy3-labelled anti-rabbit Ig, and unstained cells. The results of NIH 3T3 cells incubated (1 hour) with a carrier peptide coupled to GST (0.6-20 μM) are shown in FIG. 11. A dose-dependent increase was shown over the range up to 20 μM in intracellular GST levels (immunofluorescence detection, using a Biolumin 960 Fluorescence Reader). In the same series of experiments, little uptake of GST wild type (uncoupled GST) was detected. To confirm that the cell-associated GST was localized intracellularly rather than non-specifically associated with the extracellular membranes, standard indirect immunofluorescence microscopy techniques were used. Cells treated with GST linked to the carrier peptides, exhibited a strong, general fluorescence signal, under the field of view, associated with whole populations of cells. No fluorescence was observed with cells treated with uncoupled GST (FIG. 12). The results from the latter experiments were confirmed with confocal laser scanning microscopy to dissect the protein-treated NIH 3T3 cells. A ten-step Z-position 1 μM sectional scanning of the cells showed strongest fluorescence signals representing immunoreactive GST in the midsections of the cells. Membrane staining was negligible because much weaker fluorescence was seen at the top and the bottom of the cells. This scanning analysis demonstrated that cell-associated GST was localized intracellularly. Indirect immunofluorescence testing, carried out confocally using cells treated with wild type GST, exhibited background staining (FIG. 13). Cellular uptake of protein was also confirmed with washed NIH 3T3 cell lysates, and GST detection kit (Amersham Pharmacia Biotech). The results (not shown) indicate the GST imported into the cells was still capable of binding to an enzyme substrate, and strongly suggest that the imported protein retains a substantial degree of native confirmation. These data demonstrated that GST was not localized within intracellular compartments, such as lysosomes, where intracellular proteases are highly active.

The time and temperature dependence of protein import was further determined by analyzing the levels of imported protein, using a fluorescence readout measured on a Biolumin Fluorescence Reader as described above. In the temperature-dependence study, it was determined that uptake of GST occurred equally well at 22° C. and 37° C. However, protein import was significantly reduced at 4° C., although not entirely abolished. The kinetics of protein import was also studied. Within 30 minutes of treatment cells were positive for GST, and protein accumulation continued intracellularly up to 18 hours of incubation with the carrier peptides linked to GST (data not shown).

9. Cell Viability Studies

Cell numbers were estimated with a haemocytometer. Cell viability was assessed using a live/dead cytotoxicity kit (Molecular Probes Inc.), following the kit manufacturer's instructions. Briefly, the method involved using membrane-permeant calcein AM, cleavable by esterases in live cells to yield cytoplasmic green fluorescence, and, membrane-impermeant ethidium homodimer-1. The latter reagent labels nucleic acids of membrane compromised (dead) cells with red fluorescence. The results of the cell viability experiments were routinely confirmed with a (0.4% w/v) Trypan blue dye (Sigma) exclusion test. Cell cultures exhibited greater than 95% viability after incubation of transport peptide linked to target biomolecule. These data indicates that protein import was not cytotoxic.

1. A carrier peptide for transport of a target molecule across a cell membrane and into a cell, the carrier peptide being selected from the group consisting of:
(SEQ ID NO:1)
Thr-Lys-Lys-Pro-Leu-Pro-Pro-Thr-Pro-Glu-Glu-Asp;
(SEQ ID NO:2)
Ser-Glu-Pro-Ala-Val-Ser-Pro-Leu-Leu-Pro-Arg-Lys-Glu-Arg;
(SEQ ID NO:3)
Ala-Pro-Thr-Met-Pro-Pro-Pro-Leu-Pro-Pro-Leu-Gly-Gly-Lys;
(SEQ ID NO:4)
Ala-Pro-Thr-Arg-Val-Pro-Leu-Pro-Leu-Pro-Val-Gly-Gly-Lys;
(SEQ ID NO:5)
Ala-Pro-Thr-Arg-Ala-Val-Leu-Pro-Leu-Ala-Val-Gly-Gly-Lys;
(SEQ ID NO:6);
Ala-Pro-Thr-Arg-Val-Leu-Pro-Pro-Leu-Val-Ala-Gly-Gly-Lys;
(SEQ ID NO:7)
Ala-Pro-Thr-Arg-Val-Leu-Leu-Pro-Leu-Leu-Val-Gly-Gly-Lys;
(SEQ ID NO:8)
and Ala-Pro-Thr-Arg-Leu-Pro-Leu-Pro-Leu-Val-Ala-Gly-Gly-Lys.

2. A conjugate for delivery of a target molecule into a cell said conjugate comprising a carrier peptide according to claim 1 covalently bonded to said target molecule.

3. A conjugate according to claim 2 wherein said target molecule is selected from a reporter moiety and a biological molecule said biological molecule optionally containing a reporter moiety covalently bonded thereto.

4. A conjugate according to claim 3 wherein said reporter moiety is selected from a radioactively-labelled moiety and a luminescent molecule.

5. A conjugate according to claim 4 wherein said luminescent molecule is selected from the group consisting of fluoresceins, rhodamines, coumarins, pyrene dyes and cyanine dyes.

6. A conjugate according to claim 4 wherein said luminescent molecule is an environmentally sensitive fluorescent dye.

7. A conjugate according to claim 4 wherein said luminescent molecule is selected from the group consisting of Green fluorescent protein (GFP) and analogues thereof, a photoprotein, or a luciferase.

8. A conjugate according to any of claims 2 to 7 wherein said biological material is selected from the group consisting of antibodies, antigens, proteins, enzymes, carbohydrates, lipids, drugs, hormones and nucleotides and deoxy- or ribo-nucleic acids (such as DNA or RNA) which contain or are derivatised to contain one of amino, hydroxyl, phosphate, thiophosphoryl, sulphydryl or carboxyl groups.

9. A method for delivery of a target molecule into a cell, the method comprising the steps of:
i) providing a conjugate comprising a carrier peptide according to claim 1 covalently bonded to the target molecule; and,
ii) contacting the cell with said conjugate under conditions so as to effect delivery of the target molecule into the cell.

10. A method according to claim 9 wherein said target molecule is selected from a reporter moiety and a biological molecule said biological molecule optionally containing a reporter moiety covalently bonded thereto.

11. A method according to claim 10 wherein said reporter moiety is selected from a radioactively-labelled moiety and a luminescent molecule.

12. A method according to claim 11 wherein said luminescent molecule is selected from the group consisting of fluoresceins, rhodamines, coumarins, pyrene dyes and cyanine dyes.

13. A method according to claim 11 wherein said luminescent molecule is an environmentally sensitive fluorescent dye.

14. A method according to claim 11 wherein said luminescent molecule is selected from the group consisting of Green fluorescent protein (GFP) and analogues thereof, a photoprotein, or a luciferase.

15. A method according to any of claims 10 to 14 wherein said biological molecule is selected from the group consisting of antibodies, antigens, proteins, enzymes, carbohydrates, lipids, drugs, hormones and nucleotides and oxy- or deoxy- polynucleic acids which contain or are derivatised to contain one of amino, hydroxyl, phosphate, thiophosphoryl, sulphydryl or carboxyl groups.

16. A method for measuring a cellular process, the method comprising:
i) providing a population of cells in a fluid medium;
ii) contacting said cells with a conjugate under conditions so as to effect delivery of said conjugate into said cells said conjugate comprising a carrier peptide according to claim 1 covalently bonded to a reporter moiety or to a biological molecule said biological molecule containing a reporter moiety covalently bonded thereto; and
iii) detecting the output of the reporter moiety as a means of measuring said cellular process.

17. A method according to claim 16 further comprising contacting said cells with said conjugate in the presence of a substance whose effect on said cellular process is to be determined and detecting the output of the reporter moiety as a means of measuring said cellular process.

18. An isolated polynucleotide comprising a sequence encoding a peptide comprising the amino acid sequence according to any one of SEQ.ID No.3 to SEQ ID No.8.

19. Use of a peptide according to claim 1 or a conjugate according to any one of claims 2 to 8 to effect delivery of a target molecule into living cells.

20. A conjugate for delivery of a target molecule into a cell said conjugate comprising a carrier peptide having from 10-15 amino acids and having a core sequence of 3-5 hydrophobic amino acids flanked by flanking amino acid sequences, characterised in that said core sequence comprises residues selected from proline and leucine such that there is at least one of each of proline and leucine and that the core sequence is symmetrical about an amino acid or a bond, covalently bonded to an environmentally sensitive fluorescent dye.

21. A conjugate for delivery of a target molecule into a cell said conjugate comprising a carrier peptide having from 10-15 amino acids and having a core sequence of 3-5 hydrophobic amino acids flanked by flanking amino acid sequences, characterised in that said core sequence comprises residues selected from proline and leucine such that there is at least one of each of proline and leucine and that the core sequence is symmetrical about an amino acid or a bond, covalently bonded to a reporter wherein said reporter is a Green fluorescent protein (GFP) or analogues thereof.

22. A peptide according to claims 21 or 22 wherein said core sequence is selected from: -Pro-Leu-Pro-; Leu-Pro-Leu-; -Pro-Leu-Leu-Pro-; -Leu-Pro-Pro-Leu-; -Pro-Pro-Leu-Pro-Pro-; -Leu-Leu-Pro-Leu-Leu-; -Pro-Leu-Pro-Leu-Pro- and -Leu-Pro-Leu-Pro-Leu-.


SEQUENCE LISTING (16 sequences)

SEQ ID NO: 1 - protein, 12 aa, artificial sequence
  synthetic oligomer
TKKPLPPTPEED

SEQ ID NO: 2 - protein, 14 aa, artificial sequence
  synthetic oligomer
SEPAVSPLLPRKER

SEQ ID NO: 3 - protein, 14 aa, artificial sequence
  synthetic oligomer
APTMPPPLPPLGGK

SEQ ID NO: 4 - protein, 14 aa, artificial sequence
  synthetic oligomer
APTRVPLPLPVGGK

SEQ ID NO: 5 - protein, 14 aa, artificial sequence
  synthetic oligomer
APTRAVLPLAVGGK

SEQ ID NO: 6 - protein, 14 aa, artificial sequence
  synthetic oligomer
APTRVLPPLVAGGK

SEQ ID NO: 7 - protein, 14 aa, artificial sequence
  synthetic oligomer
APTRVLLPLLVGGK

SEQ ID NO: 8 - protein, 14 aa, artificial sequence
  synthetic oligomer
APTRLPLPLVAGGK

SEQ ID NO: 9 - DNA, 36 bp, artificial sequence
  synthetic oligomer
actaagaagcctcttcctcctactcctgaggaggat

SEQ ID NO: 10 - DNA, 42 bp, artificial sequence
  synthetic oligomer
tctgagcctgctgtttctcctcttcttcctcgtaaggagcgt

SEQ ID NO: 11 - DNA, 42 bp, artificial sequence
  synthetic oligomer
gctcctactatgcctcctcctcttcctcctcttggtggtaag

SEQ ID NO: 12 - DNA, 42 bp, artificial sequence
  synthetic oligomer
gctcctactcgtgttcctcttcctcttcctgttggtggtaag

SEQ ID NO: 13 - DNA, 42 bp, artificial sequence
  synthetic oligomer
gctcctactcgtgctgttcttcctcttgctgttggtggtaag

SEQ ID NO: 14 - DNA, 42 bp, artificial sequence
  synthetic oligomer
gctcctactcgtgttcttcctcctcttgttgctggtggtaag

SEQ ID NO: 15 - DNA, 42 bp, artificial sequence
  synthetic oligomer
gctcctactcgtgttcttcttcctcttcttgttggtggtaag

SEQ ID NO: 16 - DNA, 42 bp, artificial sequence
  synthetic oligomer
gctcctactcgtcttcctcttcctcttgttgctggtggtaag